(19) United States
(12) Patent Application Publication
(10) Pub. No.: US 2021/0186973 A1
(43) Pub. Date: Jun. 24, 2021

(54) COMBINATION OF RIBOCICLIB AND DABRAFENIB FOR TREATING OR PREVENTING CANCER

(71) Applicant: NOVARTIS AG, Basel (CH)

(72) Inventors: Giordano CAPONIGRO, Cambridge, MA (US); Thomas HORN-SPIROHN, Cambridge, MA (US); Joseph LEHAR, Cambridge, MA (US)

(21) Appl. No.: 17/074,120

(22) Filed: Oct. 19, 2020

Related U.S. Application Data
(63) Continuation of application No. 15/755,270, filed on Feb. 26, 2018, now abandoned.
(60) Provisional application No. 62/211,027, filed on Aug. 28, 2015.

(51) Int. Cl. A61K 31/519; A61K 31/4439; A61K 31/506; A61P 35/02

(52) U.S. Cl. CPC A61K 31/519; A61K 31/4439; A61K 2300/00; A61P 35/02; A61K 31/506

(57) ABSTRACT

The present disclosure relates to pharmaceutical combinations comprising a cyclin dependent kinase 4/6 (CDdK4/6) inhibitor compound, (b) a B-Raf inhibitor compound, and optionally (c) an aipha-isoform specific phosphatidylinositol 3-kinase (P13K) inhibitor compound, for the treatment or prevention of cancer, as well as related pharmaceutical compositions, uses, and methods of treatment or prevention of cancer.

### CROSS-REFERENCE TO RELATED APPLICATIONS

This application is a continuation application of U.S. application Ser. No. 15/755,270, filed 26 Feb. 2018, which is a U.S. National Phase filing of International Application Serial Number PCT/M2016/055076 filed 25 Aug. 2016, which claims the benefit of U.S. Provisional Patent Application Ser. No. 62/211,027 filed 28 Aug. 2015. The full disclosures of these applications are incorporated herein by reference in their entirety and for all purposes.

### TECHNICAL FIELD

The present disclosure relates to pharmaceutical combinations comprising (a) a cyclin dependent kinase 4/6 (CDK4/6) inhibitor compound, (b) a B-Raf inhibitor compound, and optionally (c) an alpha-isoform specific phosphatidylinositol 3-kinase (PI3K) inhibitor compound, for the treatment or prevention of cancer. The disclosure also provides related pharmaceutical compositions, uses, and methods of treatment or prevention of cancer.

### BACKGROUND

Tumor development is closely associated with genetic alteration and deregulation of cyclin dependent kinases (CDKs) and their regulators, suggesting that inhibitors of CDKs may be useful anti-cancer therapeutics. Indeed, early results suggest that transformed and normal cells differ in their requirement for, e.g., cyclin D/CDK4/6 and that it may be possible to develop novel antineoplastic agents devoid of the general host toxicity observed with conventional cytotoxic and cytostatic drugs.

The function of CDKs is to phosphorylate and thus activate or deactivate certain proteins, including, e.g., retinoblastoma proteins, lamins, histone H1, and components of the mitotic spindle. The catalytic step mediated by CDKs involves a phospho-transfer reaction from ATP to the macromolecular enzyme substrate. Several groups of compounds (reviewed in, e.g., Fischer, P. M. Curr. Opin. Drug Discovery Dev. 2001, 4, 623-634) have been found to possess anti-proliferative properties by virtue of CDK-specific ATP antagonism.

At a molecular level, mediation of CDK/cyclin complex activity requires a series of stimulatory and inhibitory phosphorylation, or dephosphorylation, events. CDK phosphorylation is performed by a group of CDK activating kinases (CAKs) and/or kinases such as wee2, Myt1 and Mik1. Dephosphorylation is performed by phosphatases such as Cdc25(a & c), PP2A, or KAP.

CDK/cyclin complex activity may be further regulated by two families of endogenous cellular proteinaceous inhibitors: the Kip/Cip family, or the INK family. The INK proteins specifically bind CDK4 and CDK6. p16^{ink4} (also known as MTS1) is a potential tumor suppressor gene that is mutated or deleted in a large number of primary cancers. The Kip/Cip family contains proteins such as p21^{Cip1,Waf1}, p27^{Kip1} and p57^{kip2}, where p21 is induced by p53 and is able to inactivate the CDK2/cyclin(E/A) complex. Atypically low levels of p27 expression have been observed in breast, colon and prostate cancers. Conversely, over-expression of cyclin E in solid tumors has been shown to correlate with poor patient prognosis. Over-expression of cyclin D1 has been associated with esophageal, breast, squamous, and non-small cell lung carcinomas.

The pivotal roles of CDKs, and their associated proteins, in coordinating and driving the cell cycle in proliferating cells have been outlined above. Some of the biochemical pathways in which CDKs play a key role have also been described. The development of monotherapies for the treatment of proliferative disorders, such as cancers, using therapeutics targeted generically at CDKs, or at specific CDKs, is therefore potentially highly desirable.

Mutations in various Ras GTPases and the B-Raf kinase have been identified that can lead to sustained and constitutive activation of the MAPK pathway, ultimately resulting in increased cell division and survival. As a consequence of this, these mutations have been strongly linked with the establishment, development, and progression of a wide range of human cancers. The biological role of the Raf kinases, and specifically that of B-Raf, in signal transduction is described in Davies, H., et al., Nature (2002) 9:1-6; Garnett, M. J. & Marais, R., Cancer Cell (2004) 6:313-319; Zebisch, A. & Troppmair, J., Cell. Mol. Life Sci. (2006) 63:1314-1330; Midgley, R. S. & Kerr, D. J., Crit. Rev. Onc/Hematol. (2002) 44:109-120; Smith, R. A., et al., Curr. Top. Med. Chem. (2006) 6:1071-1089; and Downward, J., Nat. Rev. Cancer (2003) 3:11-22.

Naturally occurring mutations of the B-Raf kinase that activate MAPK pathway signaling have been found in a large percentage of human melanomas (Davies (2002) supra) and thyroid cancers (Cohen et al J. Nat. Cancer Inst. (2003) 95(8) 625-627 and Kimura et al Cancer Res. (2003) 63(7) 1454-1457), as well as at lower, but still significant, frequencies in the following:

Barret's adenocarcinoma (Garnett et al., Cancer Cell (2004) 6 313-319 and Sommerer et al Oncogene (2004) 23(2) 554-558), billiary tract carcinomas (Zebisch et al., Cell. Mol. Life Sci. (2006) 63 1314-1330), breast cancer (Davies (2002) supra), cervical cancer (Moreno-Bueno et al Clin. Cancer Res. (2006) 12(12) 3865-3866), cholangiocarcinoma (Tannapfel et al Gut (2003) 52(5) 706-712), central nervous system tumors including primary CNS tumors such as glioblastomas, astrocytomas and ependymomas (Knobbe et al Acta Neuropathol. (Berl.) (2004) 108(6) 467-470, Davies (2002) supra, and Garnett et al., Cancer Cell (2004) supra) and secondary CNS tumors (i.e., metastases to the central nervous system of tumors originating outside of the central nervous system), colorectal cancer, including large intestinal colon carcinoma (Yuen et al Cancer Res. (2002) 62(22) 6451-6455, Davies (2002) supra and Zebisch et al., Cell. Mol. Life Sci. (2006), gastric cancer (Lee et al Oncogene (2003) 22(44) 6942-6945), carcinoma of the head and neck including squamous cell carcinoma of the head and neck (Cohen et al J. Nat. Cancer Inst. (2003) 95(8) 625-627 and Weber et al Oncogene (2003) 22(30) 4757-4759), hematologic cancers including leukemias (Garnett et al., Cancer Cell (2004) supra, particularly acute lymphoblastic leukemia (Garnett et al., Cancer Cell (2004) supra and Gustafsson et al Leukemia (2005) 19(2) 310-312), acute myelogenous leukemia (AML) (Lee et al Leukemia (2004) 18(1) 170-172, and Christiansen et al Leukemia (2005) 19(12) 2232-2240), myelodysplastic syndromes (Christiansen et al Leukemia (2005) supra) and chronic myelogenous leukemia (Mizuchi et al Biochem. Biophys. Res. Commun. (2005) 326(3) 645-651); Hodgkin's lymphoma (Figl et al Arch. Dermatol. (2007) 143(4) 495-499), non-Hodgkin's lymphoma (Lee et al Br. J. Cancer (2003) 89(10) 1958-1960), megakaryoblastic leukemia (Eychene et al Oncogene (1995) 10(6) 1159-1165) and multiple myeloma (Ng et al Br. J. Haematol. (2003) 123(4) 637-645), hepatocellular carcinoma (Garnett et al., Cancer Cell (2004), lung cancer (Brose et al Cancer Res. (2002) 62(23) 6997-7000, Cohen et al J. Nat. Cancer Inst. (2003) supra and Davies (2002) supra), including small cell lung cancer (Pardo et al EMBO J. (2006) 25(13) 3078-3088) and non-small cell lung cancer (Davies (2002) supra), ovarian cancer (Russell & McCluggage J. Pathol. (2004) 203(2) 617-619 and Davies (2002) supr), endometrial cancer (Garnett et al., Cancer Cell (2004) supra, and Moreno-Bueno et al Clin. Cancer Res. (2006) supra), pancreatic cancer (Ishimura et al Cancer Lett. (2003) 199(2) 169-173), pituitary adenoma (De Martino et al J. Endocrinol. Invest. (2007) 30(1) RC1-3), prostate cancer (Cho et al Int. J. Cancer (2006) 119(8) 1858-1862), renal cancer (Nagy et al Int. J. Cancer (2003) 106(6) 980-981), sarcoma (Davies (2002) supra), and skin cancers (Rodriguez-Viciana et al Science (2006) 311(5765) 1287-1290 and Davies (2002) supra). Overexpression of c-Raf has been linked to AML (Zebisch et al., Cancer Res. (2006) 66(7) 3401-3408, and Zebisch (Cell. Mol. Life Sci. (2006)) and erythroleukemia (Zebisch et la., Cell. Mol. Life Sci. (2006).

Phosphatidylinositol 3-kinases (PI3Ks) comprise a family of lipid kinases that catalyze the transfer of phosphate to the D-3′ position of inositol lipids to produce phosphoinositol-3-phosphate (PIP), phosphoinositol-3,4-diphosphate (PIP2) and phosphoinositol-3,4,5-triphosphate (PIP₃) that, in turn, act as second messengers in signaling cascades by docking proteins containing pleckstrin-homology, FYVE, Phox and other phospholipid-binding domains into a variety of signaling complexes often at the plasma membrane ((Vanhaesebroeck et al., Annu. Rev. Biochem 70:535 (2001); Katso et al., Annu. Rev. Cell Dev. Biol. 17:615 (2001)). Of the two Class 1 PI3Ks, Class 1A PI3Ks are heterodimers composed of a catalytic p110 subunit (α, β, δ isoforms) constitutively associated with a regulatory subunit that can be p85α, p55α, p50α, p85β or p55γ. The Class 1B sub-class has one family member, a heterodimer composed of a catalytic p110γ subunit associated with one of two regulatory subunits, p101 or p84 (Fruman et al., Annu Rev. Biochem. 67:481 (1998); Suire et al., Curr. Biol. 15:566 (2005)). The modular domains of the p85/55/50 subunits include Src Homology (SH2) domains that bind phosphotyrosine residues in a specific sequence context on activated receptor tyrosine kinases and cytoplasmic tyrosine kinases, resulting in activation and localization of Class 1A PI3Ks. Class 1B PI3K is activated directly by G protein-coupled receptors that bind a diverse repertoire of peptide and non-peptide ligands (Stephens et al., Cell 89:105 (1997)); Katso et al., Annu. Rev. Cell Dev. Biol. 17:615-675 (2001)). Consequently, the resultant phospholipid products of class I PI3K link upstream receptors with downstream cellular activities including proliferation, survival, chemotaxis, cellular trafficking, motility, metabolism, inflammatory and allergic responses, transcription and translation (Cantley et al., Cell 64:281 (1991); Escobedo and Williams, Nature 335:85 (1988); Fantl et al., Cell 69:413 (1992)).

In many cases, PIP₂ and PIP₃ recruit Akt, the product of the human homologue of the viral oncogene v-Akt, to the plasma membrane where it acts as a nodal point for many intracellular signaling pathways important for growth and survival (Fantl et al., Cell 69:413-423(1992); Bader et al., Nature Rev. Cancer 5:921 (2005); Vivanco and Sawyer, Nature Rev. Cancer 2:489 (2002)). Aberrant regulation of PI3K, which often increases survival through Akt activation, is one of the most prevalent events in human cancer and has been shown to occur at multiple levels. The tumor suppressor gene PTEN, which dephosphorylates phosphoinositides at the 3′ position of the inositol ring and in so doing antagonizes PI3K activity, is functionally deleted in a variety of tumors. In other tumors, the genes for the p110α isoform, PIK3CA, and for Akt are amplified and increased protein expression of their gene products has been demonstrated in several human cancers.

Furthermore, mutations and translocation of p85α that serve to up-regulate the p85-p110 complex have been described in human cancers. Finally, somatic missense mutations in PIK3CA that activate downstream signaling pathways have been described at significant frequencies in a wide diversity of human cancers (Kang at el., Proc. Natl. Acad. Sci. USA 102:802 (2005); Samuels et al., Science 304:554 (2004); Samuels et al., Cancer Cell 7:561-573 (2005)). These observations show that deregulation of phosphoinositol-3 kinase and the upstream and downstream components of this signaling pathway is one of the most common deregulations associated with human cancers and proliferative diseases (Parsons et al., Nature 436:792 (2005); Hennessey at el., Nature Rev. Drug Disc. 4:988-1004 (2005)).

It has been found that the 2-carboxamide cycloamino urea derivatives of the formula (III) given below have advantageous pharmacological properties and inhibit, for example, PI3K (phosphatidylinositol 3-kinase). In particular, these compounds preferably show an improved selectivity for PI3K alpha with respect to beta and/or, delta and/or gamma subtypes. Hence, the compounds of formula (III) are suitable, for example, to be used in the treatment of diseases depending on PI3 kinases (in particular PI3K alpha, such as those showing overexpression or amplification of PI3K alpha or somatic mutation of PIK3CA), especially proliferative diseases such as tumor diseases and leukemias.

Further, these compounds preferably show improved metabolic stability and hence reduced clearance, leading to improved pharmacokinetic profiles.

By virtue of the role played by the Raf family kinases in these cancers and exploratory studies with a range of preclinical and therapeutic agents, including one selectively targeted to inhibition of B-Raf kinase activity (King A. J., et al., (2006) Cancer Res. 66:11100-11105), it is generally accepted that inhibitors of one or more Raf family kinases will be useful for the treatment of cancers associated with Raf kinase.

Many cancers, particularly those carrying B-Raf mutation, B-Raf V600E mutation, PIK3CA mutation and/or PIK3CA overexpression are amenable to treatments with, for example, a B-Raf inhibitor. However, in certain cases, the cancers acquire resistance to the chosen therapeutic and ultimately become refractory to treatment.

In spite of numerous treatment options for cancer patients, there remains a need for effective and safe therapeutic agents and a need for their preferential use in combination therapy. In particular, there is a need for effective methods of treating cancers, especially those cancers that have been resistant and/or refractive to current therapies.

### SUMMARY

In a first aspect, provided herein is a pharmaceutical combination comprising:

(a) a first compound having the structure of formula (I):

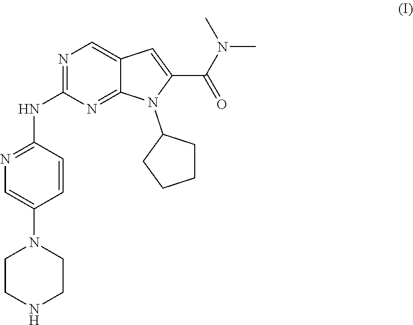 [SMILES: CN(C)C(=O)c1cc2cnc(Nc3ccc(N4CCNCC4)cn3)nc2n1C1CCCC1]

or a pharmaceutically acceptable salt or solvate thereof, and

(b) a second compound having the structure of formula (II):

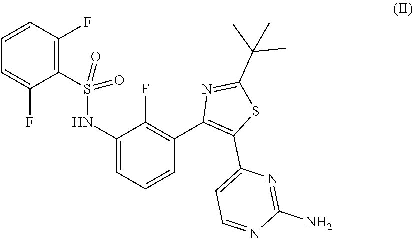 [SMILES: CC(C)(C)c1nc(-c2cccc(NS(=O)(=O)c3c(F)cccc3F)c2F)c(-c2ccnc(N)n2)s1]

or a pharmaceutically acceptable salt or solvate thereof.

In an embodiment, the compound having the structure of formula (I), or a pharmaceutically acceptable salt or solvate thereof, and the compound having the structure of formula (II), or a pharmaceutically acceptable salt or solvate thereof, are in the same formulation.

In an embodiment, the compound having the structure of formula (I), or a pharmaceutically acceptable salt or solvate thereof, and the compound having the structure of formula (II), or a pharmaceutically acceptable salt or solvate thereof, are in separate formulations.

In an embodiment, the combination of the first aspect is for simultaneous or sequential administration.

In an embodiment of the first aspect, the pharmaceutical combination further comprises a third compound having the structure of formula (III):

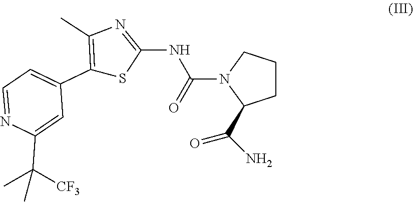 [SMILES: Cc1nc(NC(=O)N2CCC[C@H]2C(N)=O)sc1-c1ccnc(C(C)(C)C(F)(F)F)c1]

or a pharmaceutically acceptable salt or solvate thereof.

In an embodiment, the compound having the structure of formula (I), or a pharmaceutically acceptable salt or solvate thereof, the compound having the structure of formula (II), or a pharmaceutically acceptable salt or solvate thereof, and the compound having the structure of formula (III), or a pharmaceutically acceptable salt or solvate thereof, are in the same formulation.

In an embodiment, the compound having the structure of formula (I), or a pharmaceutically acceptable salt or solvate thereof, the compound having the structure of formula (II), or a pharmaceutically acceptable salt or solvate thereof, and the compound having the structure of formula (III), or a pharmaceutically acceptable salt or solvate thereof, are in 2 or more separate formulations.

In an embodiment, the compound having the structure of formula (I), or a pharmaceutically acceptable salt or solvate thereof, the compound having the structure of formula (II), or a pharmaceutically acceptable salt or solvate thereof, and the compound having the structure of formula (III), or a pharmaceutically acceptable salt or solvate thereof, are in 2 or 3 separate formulations.

In an embodiment, the pharmaceutical combination comprising the compound having the structure of formula (I), or a pharmaceutically acceptable salt or solvate thereof, the compound having the structure of formula (II), or a pharmaceutically acceptable salt or solvate thereof, and the compound having the structure of formula (III), or a pharmaceutically acceptable salt or solvate thereof is for simultaneous or sequential administration.

In a particular embodiment of the pharmaceutical combinations described supra, the first compound is the succinate salt of the compound having the structure of formula (I).

In a particular embodiment of the pharmaceutical combinations described supra, the second compound is the mesylate salt of the compound having the structure of formula (II).

In a second aspect, provided herein is a method for the treatment or prevention of cancer in a subject in need thereof, comprising administering to the subject a therapeutically effective amount of a pharmaceutical combination according to any one of the embodiments described supra.

In an embodiment, the cancer is selected from the group consisting of melanoma, lung cancer (including non-small-cell lung cancer (NSCLC)), colorectal cancer (CRC), breast cancer, kidney cancer, renal cell carcinoma (RCC), liver cancer, acute myelogenous leukemia (AML), myelodysplastic syndromes (MDS), thyroid cancer, pancreatic cancer, neurofibromatosis and hepatocellular carcinoma.

In a particular embodiment, the cancer is colorectal cancer.

In certain particular embodiments of the second aspect, the cancer is characterized by one or more of a B-Raf mutation, B-Raf V600E mutation, PIK3CA mutation and PIK3CA overexpression.

In a third aspect, provided herein is a pharmaceutical combination as described supra for use in the treatment or prevention of cancer.

In a fourth aspect, provided herein is a pharmaceutical combination as described supra for use in the manufacture of a medicament for the treatment or prevention of cancer.

In certain embodiments of the third and fourth aspects, the cancer is selected from the group consisting of melanoma, lung cancer (including non-small-cell lung cancer (NSCLC)), colorectal cancer (CRC), breast cancer, kidney cancer, renal cell carcinoma (RCC), liver cancer, acute myelogenous leukemia (AML), myelodysplastic syndromes (MDS), thyroid cancer, pancreatic cancer, neurofibromatosis and hepatocellular carcinoma.

In a particular embodiment, the cancer is colorectal cancer.

In certain particular embodiments of the third and fourth aspects, the cancer is characterized by one or more of a B-Raf mutation, B-Raf V600E mutation, PIK3CA mutation and PIK3CA overexpression.

In a fifth aspect, provided herein is the use of a pharmaceutical combination as described supra for the manufacture of a medicament for the treatment or prevention of cancer.

In a sixth aspect, provided herein is the use of a pharmaceutical combination as described supra for the treatment or prevention of cancer.

In particular embodiments of the fifth and sixth aspects, the cancer is selected from the group consisting of melanoma, lung cancer (including non-small-cell lung cancer (NSCLC)), colorectal cancer (CRC), breast cancer, kidney cancer, renal cell carcinoma (RCC), liver cancer, acute myelogenous leukemia (AML), myelodysplastic syndromes (MDS), thyroid cancer, pancreatic cancer, neurofibromatosis and hepatocellular carcinoma.

In a particular embodiment, the cancer is colorectal cancer.

In certain particular embodiments of the fifth and sixth aspects, the cancer is characterized by one or more of a B-Raf mutation, B-Raf V600E mutation, PIK3CA mutation and PIK3CA overexpression.

In a seventh aspect, provided herein is a pharmaceutical composition comprising:

(a) a first compound having the structure of formula (I):

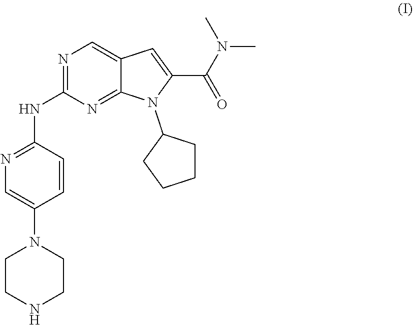 [SMILES: CN(C)C(=O)c1cc2cnc(Nc3ccc(N4CCNCC4)cn3)nc2n1C1CCCC1]

or a pharmaceutically acceptable salt or solvate thereof, and

(b) a second compound having the structure of formula (II):

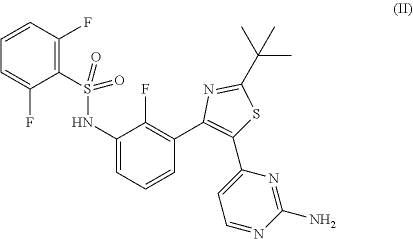 [SMILES: CC(C)(C)c1nc(-c2cccc(NS(=O)(=O)c3c(F)cccc3F)c2F)c(-c2ccnc(N)n2)s1]

or a pharmaceutically acceptable salt or solvate thereof.

In an embodiment of the seventh aspect, the pharmaceutical composition further comprises a third compound having the structure of formula (III):

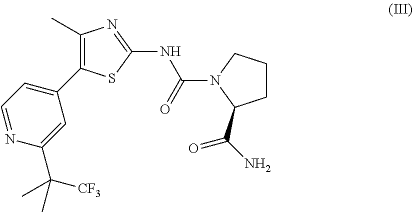 [SMILES: Cc1nc(NC(=O)N2CCC[C@H]2C(N)=O)sc1-c1ccnc(C(C)(C)C(F)(F)F)c1]

or a pharmaceutically acceptable salt or solvate thereof.

In an embodiment, the pharmaceutical composition comprises one or more excipients.

### BRIEF DESCRIPTION OF THE DRAWINGS

FIG. 1 shows dose-response curves for LEE011, dabrafenib, BYL719, and combinations thereof over 6 B-Raf mutant colorectal cancer cell lines. The x-axis indicates the log10 of the treatment dilution; the y-axis indicates the cell count after treatment relative to DMSO. The strong dashed line indicates the number of cells before the start of the treatment (‘baseline’).

FIG. 2 shows maximum Caspase 3/7 induction for LEE011, dabrafenib, BYL719, and combinations thereof in 6 B-Raf mutant colorectal cancer cell lines and after 24 h, 48 h, and 72 h (different shades of grey). The x-axis indicates the treatment; the y-axis indicates the maximum Caspase 3/7 induction (% of cells) seen for each treatment.

FIG. 3 shows dose-response curves for LEE011, dabrafenib, and the combination of LEE011 and dabrafenib over 6 B-Raf mutant colorectal cancer cell lines. The x-axis indicates the log10 of the treatment dilution; the y-axis indicates the cell count after treatment relative to DMSO. The strong dashed line indicates the number of cells before the start of the treatment (‘baseline’).

FIG. 4 shows maximum Caspase 3/7 induction for LEE011, dabrafenib, and the combination of LEE011 and dabrafenib in 6 colorectal cancer cell lines and after 24 h, 48 h, and 72 h (different shades of grey). The x-axis indicates the treatment; the y-axis indicates the maximum Caspase 3/7 induction (% of cells) seen for each treatment.

### DETAILED DESCRIPTION

### Inhibitor Compounds

The CDK 4/6 inhibitor 7-Cyclopentyl-2-(5-piperazin-1-yl-pyridin-2-ylamino)-7H-pyrrolo[2,3-d]pyrimidine-6-carboxylic acid dimethylamide (also known as “LEE011” or “ribociclib”) is referred to herein as the compound having the structure of formula (I), or compound (I):

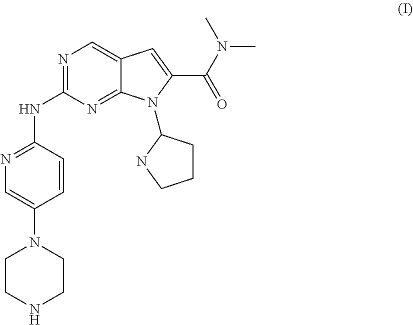 [SMILES: CN(C)C(=O)c1cc2cnc(Nc3ccc(N4CCNCC4)cn3)nc2n1C1CCCN1]

Compound (I), and pharmaceutically acceptable salts and solvates thereof are described in International Publication No. WO 2010/020675 (e.g., in Example 74), the entire contents of which is hereby incorporated by reference.

The B-Raf inhibitor N-{3-[5-(2-Amino-4-pyrimidinyl)-2-(1,1-dimethylethyl)-1,3-thiazol-4-yl]-2-fluorophenyl}-2,6-difluorobenzenesulfonamide (also known as “dabrafenib”) is referred to herein as the compound having the structure of formula (II), or compound (II):

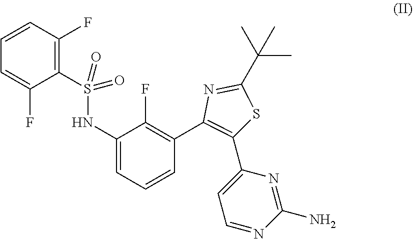 [SMILES: CC(C)(C)c1nc(-c2cccc(NS(=O)(=O)c3c(F)cccc3F)c2F)c(-c2ccnc(N)n2)s1]

Compound (II), and pharmaceutically acceptable salts and solvates thereof are described in International Publication WO 2009/137391 (e.g., Examples 58a-58e). This publication is hereby incorporated by reference in its entirety. Compound (II) may be prepared according to the methods of Example 3.

The alpha-isoform specific PI3K inhibitor compound (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (also known as “BYL719” or “alpelisib”) is referred to herein as the compound having the structure of formula (III) , or compound (III):

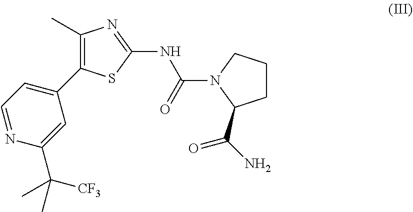 [SMILES: Cc1nc(NC(=O)N2CCC[C@H]2C(N)=O)sc1-c1ccnc(C(C)(C)C(F)(F)F)c1]

Compound (III), and pharmaceutically acceptable salts and solvates thereof are described in International Application No. WO 2010/029082 (e.g., Example 15). This publication is incorporated herein by reference in its entirety.

### Salts and Solvates

Salts of the inhibitor compounds described herein can be present alone or in a mixture with the free base form, and are preferably pharmaceutically acceptable salts. A “pharmaceutically acceptable salt”, as used herein, unless otherwise indicated, includes salts of acidic and basic groups which may be present in the compounds of the present invention. Such salts may be formed, for example, as acid addition salts, preferably with organic or inorganic acids, upon reaction with a basic nitrogen atom. Suitable inorganic acids are, for example, halogen acids, such as hydrochloric acid, sulfuric acid, or phosphoric acid. Suitable organic acids are, e.g., carboxylic acids or sulfonic acids, such as fumaric acid or methansulfonic acid. For isolation or purification purposes it is also possible to use pharmaceutically unacceptable salts, for example picrates or perchlorates.

In a preferred embodiment of the pharmaceutical combinations described herein, the compound having the structure of formula (I) is in the form of a succinate salt.

In a preferred embodiment of the pharmaceutical combinations described herein, the compound having the structure of formula (II) is in the form of a mesylate salt.

In a preferred embodiment of the pharmaceutical combinations described herein, the compound having the structure of formula (III) is in the form of its free base.

For therapeutic use, only pharmaceutically acceptable salts, solvates or free compounds are employed (where applicable in the form of pharmaceutical preparations), and these are therefore preferred. In view of the close relationship between the compounds in their free form and those in the form of their salts, including those salts that can be used as intermediates, for example in the purification or identification of the novel compounds, any reference to the free compounds hereinbefore and hereinafter is to be understood as referring also to the corresponding salts, as appropriate and expedient. Salts contemplated herein are preferably pharmaceutically acceptable salts; suitable counter-ions forming pharmaceutically acceptable salts are known in the field.

### Methods of Treatment

The present invention invention relates to the treatment or prevention of cancer.

In an embodiment, the cancer is selected from the group consisting of melanoma, lung cancer (including non-small-cell lung cancer (NSCLC)), colorectal cancer (CRC), breast cancer, kidney cancer, renal cell carcinoma (RCC), liver cancer, acute myelogenous leukemia (AML), myelodysplastic syndromes (MDS), thyroid cancer, pancreatic cancer, neurofibromatosis and hepatocellular carcinoma.

In a particular embodiment, the cancer is colorectal cancer.

In certain particular embodiments of the second aspect, the cancer is characterized by one or more of a B-Raf mutation, B-Raf V600E mutation, PIK3CA mutation and PIK3CA overexpression.

In a third aspect, provided herein is a pharmaceutical combination as described supra for use in the treatment or prevention of cancer.

In a fourth aspect, provided herein is a pharmaceutical combination as described supra for use in the manufacture of a medicament for the treatment or prevention of cancer.

In certain embodiments of the third and fourth aspects, the cancer is selected from the group consisting of melanoma, lung cancer (including non-small-cell lung cancer (NSCLC)), colorectal cancer (CRC), breast cancer, kidney cancer, renal cell carcinoma (RCC), liver cancer, acute myelogenous leukemia (AML), myelodysplastic syndromes (MDS), thyroid cancer, pancreatic cancer, neurofibromatosis and hepatocellular carcinoma.

In a particular embodiment, the cancer is colorectal cancer.

In certain particular embodiments of the third and fourth aspects, the cancer is characterized by one or more of a B-Raf mutation, B-Raf V600E mutation, PIK3CA mutation and PIK3CA overexpression.

In a fifth aspect, provided herein is the use of a pharmaceutical combination as described supra for the manufacture of a medicament for the treatment or prevention of cancer.

In a sixth aspect, provided herein is the use of a pharmaceutical combination as described supra for the treatment or prevention of cancer.

In particular embodiments of the fifth and sixth aspects, the cancer is selected from the group consisting of melanoma, lung cancer (including non-small-cell lung cancer (NSCLC)), colorectal cancer (CRC), breast cancer, kidney cancer, renal cell carcinoma (RCC), liver cancer, acute myelogenous leukemia (AML), myelodysplastic syndromes (MDS), thyroid cancer, pancreatic cancer, neurofibromatosis and hepatocellular carcinoma.

In a particular embodiment, the cancer is colorectal cancer.

In certain particular embodiments of the fifth and sixth aspects, the cancer is characterized by one or more of a B-Raf mutation, B-Raf V600E mutation, PIK3CA mutation and PIK3CA overexpression.

### Pharmaceutical Combinations and Compositions

The combinations and compositions can be administered to a system comprising cells or tissues, as well as a human subject (e.g., a patient) or an animal subject.

The combination and composition of the present invention can be administered in various dosage forms and strength, in a pharmaceutically effective amount or a clinically effective amount.

The pharmaceutical compositions for separate administration of both combination components, or for the administration in a fixed combination, e.g., a single galenical composition comprising the combination, may be prepared in any manner known in the art and are those suitable for enteral, such as oral or rectal, and parenteral administration to mammals (warm-blooded animals), including humans.

The pharmaceutical compositions described herein may contain, from about 0.1% to about 99.9%, preferably from about 1% to about 60%, of the therapeutic agent(s). Suitable pharmaceutical compositions for the combination therapy for enteral or parenteral administration are, for example, those in unit dosage forms, such as sugar-coated tablets, tablets, capsules or suppositories, or ampoules. If not indicated otherwise, these are prepared in a manner known per se, for example by means of various conventional mixing, comminution, direct compression, granulating, sugar-coating, dissolving, lyophilizing processes, or fabrication techniques readily apparent to those skilled in the art. It will be appreciated that the unit content of a combination partner contained in an individual dose of each dosage form need not in itself constitute an effective amount since the necessary effective amount may be reached by administration of a plurality of dosage units.

A unit dosage form containing the combination of agents or individual agents of the combination of agents may be in the form of micro-tablets enclosed inside a capsule, e.g., a gelatin capsule. For this, a gelatin capsule as is employed in pharmaceutical formulations can be used, such as the hard gelatin capsule known as CAPSUGEL, available from Pfizer.

The unit dosage forms of the present invention may optionally further comprise additional conventional carriers or excipients used for pharmaceuticals. Examples of such carriers include, but are not limited to, disintegrants, binders, lubricants, glidants, stabilizers, and fillers, diluents, colorants, flavours and preservatives. One of ordinary skill in the art may select one or more of the aforementioned carriers with respect to the particular desired properties of the dosage form by routine experimentation and without any undue burden. The amount of each carriers used may vary within ranges conventional in the art. The following references which are all hereby incorporated by reference disclose techniques and excipients used to formulate oral dosage forms. See The Handbook of Pharmaceutical Excipients, 4^{th} edition, Rowe et al., Eds., American Pharmaceuticals Association (2003); and Remington: the Science and Practice of Pharmacy, 20^{th} edition, Gennaro, Ed., Lippincott Williams & Wilkins (2003).

As used herein, the term “pharmaceutically acceptable excipient” or “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289- 1329). Except insofar as any conventional carrier is incompatible with the active ingredient, its use in the therapeutic or pharmaceutical compositions is contemplated.

These optional additional conventional carriers may be incorporated into the oral dosage form either by incorporating the one or more conventional carriers into the initial mixture before or during granulation or by combining the one or more conventional carriers with granules comprising the combination of agents or individual agents of the combination of agents in the oral dosage form. In the latter embodiment, the combined mixture may be further blended, e.g., through a V-blender, and subsequently compressed or molded into a tablet, for example a monolithic tablet, encapsulated by a capsule, or filled into a sachet.

Examples of pharmaceutically acceptable disintegrants include, but are not limited to, starches; clays; celluloses; alginates; gums; cross-linked polymers, e.g., cross-linked polyvinyl pyrrolidone or crospovidone, e.g., POLYPLASDONE XL from International Specialty Products (Wayne, N.J.); cross-linked sodium carboxymethylcellulose or croscarmellose sodium, e.g., AC-DI-SOL from FMC; and cross-linked calcium carboxymethylcellulose; soy polysaccharides; and guar gum. The disintegrant may be present in an amount from about 0% to about 10% by weight of the composition. In one embodiment, the disintegrant is present in an amount from about 0.1% to about 5% by weight of composition.

Examples of pharmaceutically acceptable binders include, but are not limited to, starches; celluloses and derivatives thereof, for example, microcrystalline cellulose, e.g., AVICEL PH from FMC (Philadelphia, Pa.), hydroxypropyl cellulose hydroxylethyl cellulose and hydroxylpropylmethyl cellulose METHOCEL from Dow Chemical Corp. (Midland, Mich.); sucrose; dextrose; corn syrup; polysaccharides; and gelatin. The binder may be present in an amount from about 0% to about 50%, e.g., 2-20% by weight of the composition.

Examples of pharmaceutically acceptable lubricants and pharmaceutically acceptable glidants include, but are not limited to, colloidal silica, magnesium trisilicate, starches, talc, tribasic calcium phosphate, magnesium stearate, aluminum stearate, calcium stearate, magnesium carbonate, magnesium oxide, polyethylene glycol, powdered cellulose and microcrystalline cellulose. The lubricant may be present in an amount from about 0% to about 10% by weight of the composition. In one embodiment, the lubricant may be present in an amount from about 0.1% to about 1.5% by weight of composition. The glidant may be present in an amount from about 0.1% to about 10% by weight.

Examples of pharmaceutically acceptable fillers and pharmaceutically acceptable diluents include, but are not limited to, confectioner's sugar, compressible sugar, dextrates, dextrin, dextrose, lactose, mannitol, microcrystalline cellulose, powdered cellulose, sorbitol, sucrose and talc. The filler and/or diluent, e.g., may be present in an amount from about 0% to about 80% by weight of the composition.

The optimal dosage of each combination partner for treatment or prevention of cancer can be determined empirically for each individual using known methods and will depend upon a variety of factors, including, though not limited to, the degree of advancement of the disease; the age, body weight, general health, gender and diet of the individual; the time and route of administration; and other medications the individual is taking. Optimal dosages may be established using routine testing and procedures that are well known in the art.

The amount of each combination partner that may be combined with the carrier materials to produce a single dosage form will vary depending upon the individual treated and the particular mode of administration. In some embodiments the unit dosage forms containing the combination of agents as described herein will contain the amounts of each agent of the combination that are typically administered when the agents are administered alone.

The effective dosage of each of the combination partners employed in the combination of the invention may vary depending on the particular compound or pharmaceutical composition employed, the mode of administration, the condition being treated, and the severity of the condition being treated. Thus, the dosage regimen of the combinations described herein are selected in accordance with a variety of factors including the route of administration and the renal and hepatic function of the patient.

The effective dosage of each of the combination partners may require more frequent administration of one of the compound(s) as compared to the other compound(s) in the combination. Therefore, to permit appropriate dosing, packaged pharmaceutical products may contain one or more dosage forms that contain the combination of compounds, and one or more dosage forms that contain one of the combination of compounds, but not the other compound(s) of the combination.

Compound (I) (“LEE011”), in general, is administered in a dose in the range from 10 mg to 2000 mg per day in human. in human. In one embodiment, LEE011 is administered 600mg QD. In another embodiment, LEE011 is administered 300mg QD. In another embodiment,

LEE011 is administered in 900 mg QD.

Compound (II) (“dabrafenib”) (based on weight of the unsalted/unsolvated compound) is amnistered in a dose in the range from 20 mg to 600 mg per day in human. In one embodiment, dabrafenib is administered 100 mg to 300 mg QD. In another embodiment, dabrafenib is administered 150 mg QD.

Compound (III) (“BYL719”) may be orally administered at an effective daily dose of about 1 to 6.5 mg/kg in human adults or children. Compound (III) may be orally administered to a 70 kg body weight human adult at a daily dosage of about 70 mg to 455 mg, e.g., about 200 to 400 mg, or about 240 mg to 400 mg, or about 300 mg to 400 mg, or about 350 mg to 400 mg, in a single dose or in divided doses up to four times a day. Preferably, compound (III) is administered to a 70 kg body weight human adult at a daily dosage of about 350 mg to about 400 mg.

The optimum ratios, individual and combined dosages, and concentrations of the combination partners of the combination of the invention (i.e., Compound (I), Compound (II), and optionally Compound (III)) that yield efficacy without toxicity are based on the kinetics of the therapeutic agents' availability to target sites, and are determined using methods known to those of skill in the art.

Frequency of dosage may vary depending on the compound used and the particular condition to be treated or prevented. In general, the use of the minimum dosage that is sufficient to provide effective therapy is preferred. Patients may generally be monitored for therapeutic effectiveness using assays suitable for the condition being treated or prevented, which will be familiar to those of ordinary skill in the art.

In certain aspects, the pharmaceutical combinations described herein are useful for the treatment or prevention of cancer, or for the preparation of a medicament for the treatment or prevention of cancer. In a particular embodiment, the pharmaceutical combinations described herein are useful for the treatment of cancer, or for the preparation of a medicament for the treatment of cancer.

In certain aspects, a method for the treatment or prevention of cancer (e.g., for the treatment of cancer) is provided, comprising administering to a patient in need thereof a pharmaceutically effective amount of a pharmaceutical combination described herein.

The nature of cancer is multifactorial. Under certain circumstances, drugs with different mechanisms of action may be combined. However, just considering any combination of therapeutic agents having different mode of action does not necessarily lead to combinations with advantageous effects.

The administration of a pharmaceutical combination as described herein may result not only in a beneficial effect, e.g., a synergistic therapeutic effect, e.g., with regard to alleviating, delaying progression of or inhibiting the symptoms, but also in further surprising beneficial effects, e.g., fewer side-effects, a more durable response, an improved quality of life or a decreased morbidity, compared with a monotherapy applying only one of the pharmaceutically therapeutic agents used in the combination of the invention.

A further benefit is that lower doses of the therapeutic agents of a pharmaceutical combination as described herein can be used, for example, such that the dosages may not only often be smaller, but are also may be applied less frequently, or can be used in order to diminish the incidence of side-effects observed with one of the combination partners alone. This is in accordance with the desires and requirements of the patients to be treated.

It can be shown by established test models that a pharmaceutical combination as described herein results in the beneficial effects described herein before. The person skilled in the art is fully enabled to select a relevant test model to prove such beneficial effects. The pharmacological activity of a combination of the invention may, for example, be demonstrated in a clinical study or in an animal model.

Determining a synergistic interaction between one or more components, the optimum range for the effect and absolute dose ranges of each component for the effect may be definitively measured by administration of the components over different w/w ratio ranges and doses to patients in need of treatment. For humans, the complexity and cost of carrying out clinical studies on patients may render impractical the use of this form of testing as a primary model for synergy. However, the observation of synergy in certain experiments (see, e.g., examples 1 and 2) can be predictive of the effect in other species and animal models exist to further measure a synergistic effect. The results of such studies can also be used to predict effective dose ratio ranges and the absolute doses and plasma concentrations.

In an embodiment, the combinations and/or compositions provided herein display a synergistic effect.

In an embodiment, provided herein is a synergistic combination for administration to a human, said combination comprising the inhibitors described herein, where the dose range of each inhibitor corresponds to the synergistic ranges suggested in a suitable tumor model or clinical study.

When the combination partners, which are employed in the combination of the invention, are applied in the form as marketed as single drugs, their dosage and mode of administration can be in accordance with the information provided on the package insert of the respective marketed drug, if not mentioned herein otherwise.

### Definitions

Certain terms used herein are described below. Compounds are described using standard nomenclature. Unless defined otherwise, all technical and scientific terms used herein have the meaning that is commonly understood by one of skill in the art to which the present disclosure belongs.

The term “pharmaceutical composition” is defined herein to refer to a mixture or solution containing at least one therapeutic agent to be administered to a subject, e.g., a mammal or human, in order to prevent or treat a particular disease or condition affecting the mammal or human.

The term “pharmaceutically acceptable” is defined herein to refer to those compounds, materials, compositions and/or dosage forms, which are, within the scope of sound medical judgment, suitable for contact with the tissues a subject, e.g., a mammal or human, without excessive toxicity, irritation allergic response and other problem complications commensurate with a reasonable benefit/ risk ratio.

The term “treating” or “treatment” as used herein comprises a treatment relieving, reducing or alleviating at least one symptom in a subject or effecting a delay of progression of a disease. For example, treatment can be the diminishment of one or several symptoms of a disorder or complete eradication of a disorder, such as cancer. Within the meaning of the present invention, the term “treat” also denotes to arrest, delay the onset (i.e., the period prior to clinical manifestation of a disease) and/or reduce the risk of developing or worsening a disease. The term “prevent”, “preventing” or “prevention” as used herein comprises the prevention of at least one symptom associated with or caused by the state, disease or disorder being prevented.

The term “pharmaceutically effective amount” or “clinically effective amount” of a combination of therapeutic agents is an amount sufficient to provide an observable improvement over the baseline clinically observable signs and symptoms of the disorder treated with the combination.

The term “combination,” “therapeutic combination,” or “pharmaceutical combination” as used herein refer to either a fixed combination in one dosage unit form, or non-fixed combination or a kit of parts for the combined administration where two or more therapeutic agents may be administered independently, at the same time, or separately within time intervals, especially where these time intervals allow that the combination partners to show a cooperative, e.g., synergistic, effect.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single formulation having a fixed ratio of active ingredients or in separate formulations (e.g., capsules and/or intravenous formulations) for each active ingredient. In addition, such administration also encompasses use of each type of therapeutic agent in a sequential or separate manner, either at approximately the same time or at different times. Regardless of whether the active ingredients are administered as a single formulation or in separate formulations, the therapeutic agents are administered to the same patient as part of the same course of therapy. In any case, the treatment regimen will provide beneficial effects in treating the conditions or disorders described herein.

The term “synergistic effect” as used herein refers to action of two therapeutic agents such as, for example, the CDK inhibitor LEE011, and the B-Raf inhibitor dabrafenib, and optionally the PI3K inhibitor BYL719, producing an effect, for example, slowing the symptomatic progression of a proliferative disease, particularly cancer, or symptoms thereof, which is greater than the simple addition of the effects of each therapeutic agent administered alone. A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

The term “subject” or “patient” as used herein includes animals, which are capable of suffering from or afflicted with a cancer or any disorder involving, directly or indirectly, a cancer. Examples of subjects include mammals, e.g., humans, dogs, cows, horses, pigs, sheep, goats, cats, mice, rabbits, rats and transgenic non-human animals. In the preferred embodiment, the subject is a human, e.g., a human suffering from, at risk of suffering from, or potentially capable of suffering from cancer.

The terms “fixed combination” and “fixed dose” and “single formulation” as used herein refer to single carrier or vehicle or dosage forms formulated to deliver an amount, which is jointly therapeutically effective for the treatment of cancer, of two or more therapeutic agents to a patient. The single vehicle is designed to deliver an amount of each of the agents, along with any pharmaceutically acceptable carriers or excipients. In some embodiments, the vehicle is a tablet, capsule, pill, or a patch. In other embodiments, the vehicle is a solution or a suspension.

The term “non-fixed combination,” “kit of parts,” and “separate formulations” means that the active ingredients, e.g., LEE011 and dabrafenib are both administered to a patient as separate entities either simultaneously, concurrently or sequentially with no specific time limits, wherein such administration provides therapeutically effective levels of the two compounds in the body of the warm-blooded animal in need thereof. The latter also applies to cocktail therapy, e.g., the administration of three or more active ingredients.

The term “unit dose” is used herein to mean simultaneous administration of two or three agents together, in one dosage form, to the patient being treated. In some embodiments, the unit dose is a single formulation. In certain embodiments, the unit dose includes one or more vehicles such that each vehicle includes an effective amount of at least one of the agents along with pharmaceutically acceptable carriers and excipients. In some embodiments, the unit dose is one or more tablets, capsules, pills, injections, infusions, patches, or the like, administered to the patient at the same time.

An “oral dosage form” includes a unit dosage form prescribed or intended for oral administration.

The terms “comprising” and “including” are used herein in their open-ended and non-limiting sense unless otherwise noted.

The terms “a” and “an” and “the” and similar references in the context of describing the invention (especially in the context of the following claims) are to be construed to cover both the singular and the plural, unless otherwise indicated herein or clearly contradicted by context. Where the plural form is used for compounds, salts, and the like, this is taken to mean also a single compound, salt, or the like.

The term “about” or “approximately” shall have the meaning of within 10%, more preferably within 5%, of a given value or range.

### EXAMPLES

### Materials and Methods

The compounds were dissolved in 100% DMSO (Sigma, Catalog number D2650) at concentrations of 20 mM and stored at −20° C. until use. Compounds were arrayed in drug master plates (Greiner, Catalog number 788876) and serially diluted 3-fold (7 steps) at 2000× concentration.

Colorectal cancer cell lines used for this study were obtained, cultured and processed from commercial vendors ATCC, CellBank Australia, and HSRRB (Table 1). All cell line media were supplemented with 10% FBS (HyClone, Catalog number SH30071.03). Media for LIM2551 was additionally supplemented with 0.6 μg/mL Insulin (SIGMA, Catalog number 19278), 1 μg/mL Hydrocortisone (SIGMA, Catalog number H0135), and 10 μM 1-Thioglycerol (SIGMA, Catalog number M6145).

TABLE 1
Cell line information

| | Driver | | Source Cat | | Medium | Medium | | |
|---|---|---|---|---|---|---|---|---|
| Cell line | mutations | Source | Num | Medium | Vendor | Cat Num | #Cells | Treatment [h] |
| RKO | BRAF, PIK3CA | ATCC | CRL-2577 | EMEM | ATCC | 30-2003 | 500 | 72 |
| LIM2551 | BRAF, PIK3CA | CellBank | CBA-0170 | RPMI | ATCC | 30-2001 | 1000 | 72 |
| | | Australia | | | | | | |
| HT-29 | BRAF, PIK3CA | ATCC | HTB-38 | McCoy's 5A | ATCC | 30-2007 | 800 | 72 |
| LS411N | BRAF | ATCC | CRL-2159 | RPMI | ATCC | 30-2001 | 900 | 72 |
| COLO-205 | BRAF | ATCC | CCL-222 | RPMI | ATCC | 30-2001 | 800 | 72 |
| OUMS-23 | BRAF | HSRRB | JCRB1022 | DMEM | ATCC | 30-2002 | 900 | 72 |

Cell lines were cultured in 37° C. and 5% CO₂ incubator and expanded in T-75 flasks. In all cases cells were thawed from frozen stocks, expanded through ≥1 passage using 1:3 dilutions, counted and assessed for viability using a ViCell counter (Beckman-Coulter) prior to plating. To split and expand cell lines, cells were dislodged from flasks using 0.25% Trypsin-EDTA (GIBCO, Catalog number 25200). All cell lines were determined to be free of mycoplasma contamination as determined by a PCR detection methodology performed at Idexx Radil (Columbia, Mo., USA) and correctly identified by detection of a panel of SNPs.

Images were analyzed after adapting previously described methods (Horn, Sandmann et al. 2011) and using the Bioconductor package EBImage in R (Pau, Fuchs et al. 2010). Objects in both channels, DAPI (for Hoechst/DNA) and FITC (for Caspase 3/7), were segmented separately by adaptive thresholding and counted. A threshold for Caspase 3/7 positive objects was defined manually per cell line after comparing negative controls (DMSO) and positive controls (Staurosporine). By analyzing 17 additional object/nuclei features in the DNA channel (shape and intensity features) debris/fragmented nuclei were identified. To this end, per cell line the distributions of the additional features between positive controls (Staurosporine) and negative controls (DMSO) were compared manually. Features that could differentiate between the conditions (e.g., a shift in the distribution of a feature measurement comparing DMSO with Staurosporine) where used to define the ‘debris’ population versus the population of ‘viable’ nuclei. The debris counts were subtracted from raw nuclei counts. The resulting nuclei number was used as measure of cell proliferation (cell count').

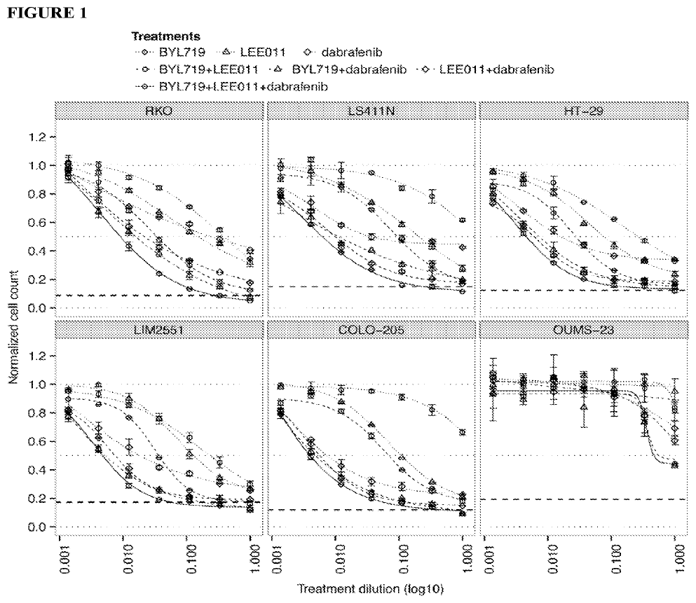

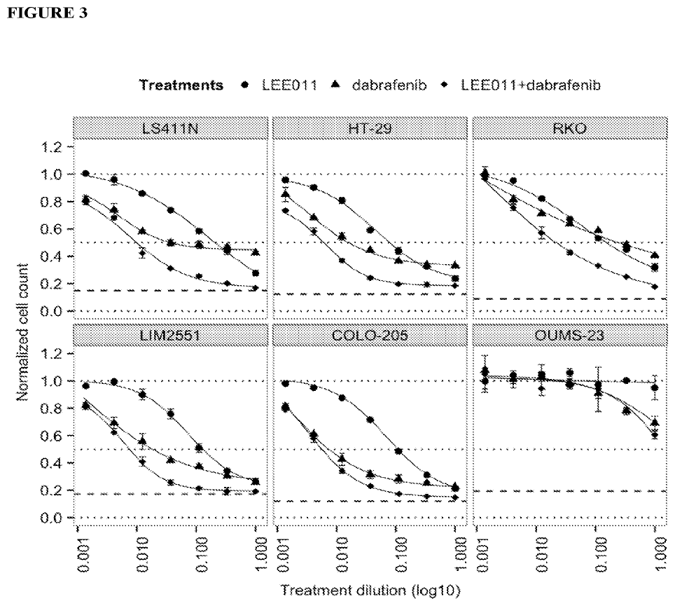

The compound's effect on cell proliferation was calculated from the cell counts of the treatments relative to the cell counts of the negative control (DMSO), in FIG. 1 and FIG. 3 denoted as ‘Normalized cell count’ (=‘xnorm’) on the y-axis. Synergistic combinations were identified using the highest single agent model (HSA) as null hypothesis (Berenbaum 1989). Excess over the HSA model predicts a functional connection between the inhibited targets (Lehar, Zimmermann et al. 2007, Lehar, Krueger et al. 2009). The model input were inhibition values per drug dose:

I=1−xnorm
I: inhibition
xnorm: normalized cell count (median of three replicates)

At every dose point of the combination treatment the difference between the inhibition of the combination and the inhibition of the stronger of the two single agents was calculated (=model residuals). Similarly, to assess the synergy of triple combinations at every dose point the difference between the inhibition of the drug triple and the inhibition of the strongest drug pair was calculated. To favor combination effects at high inhibition the residuals were weighted with the observed inhibition at the same dose point. The overall combination score C of a drug combination is the sum of the weighted residuals over all concentrations:

C=Σ_{Conc}(I_{data}*(I_{data}−I_{model}))
I_{data}: measured inhibition
I_{model}: inhibition according to HSA null hypothesis

Robust combination z-scores (zc) were calculated as the ratio of the treatments' combination scores C and the median absolute deviation (mad) of non-interacting combinations:

z_{C}=C/mad(C_{zero})
C_{zero}: combination scores of non-interacting combinations
z_{C} is an indicator for the strength of the combination with:
z_{C}≥3: synergy
3>z_{C}≥2: weak synergy
z_{C}<2: no synergy

IC50 is the oncentration that results in 50% of the cell counts relative to DMSO. IC50 calculations (see Table 2 and Table 3) were done using the DRC package in R (Ritz and Streibig 2005) and fitting a four-parameter log-logistic function to the data.

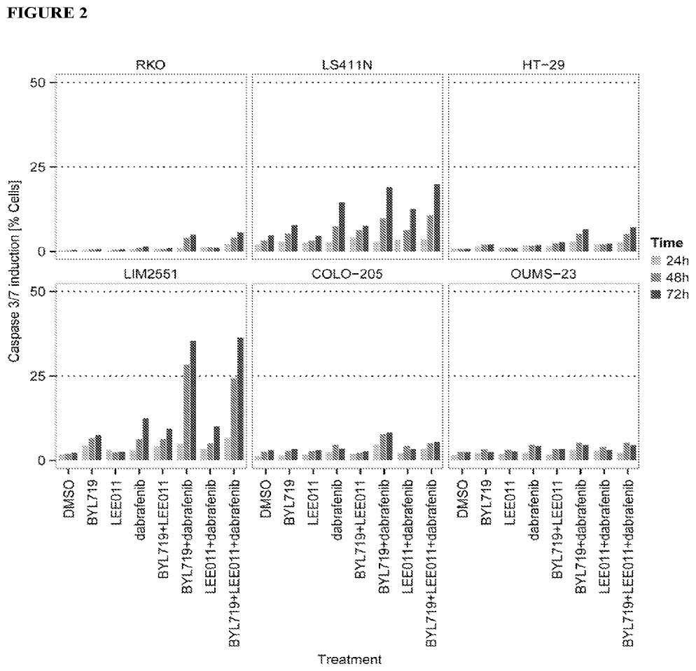

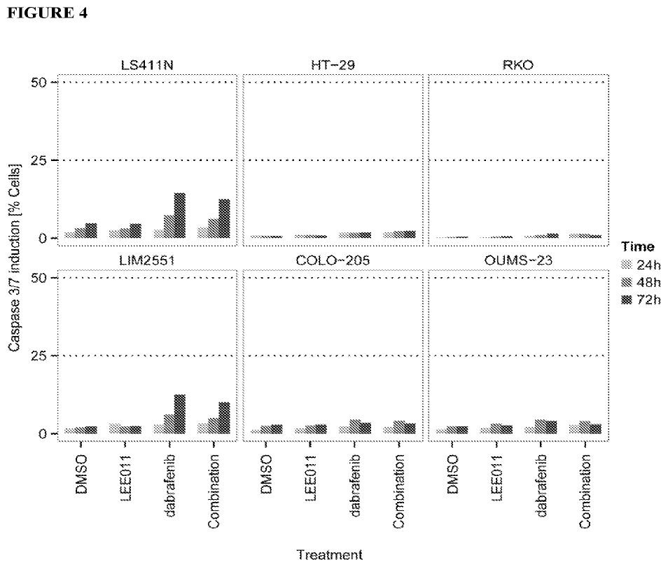

The compound's effect on apoptosis was determined by calculating the percentage of cells with activated Caspase 3/7 per treatment and time point relative to the raw cell counts (before subtraction of debris) (y-axis in FIG. 2 and FIG. 4). Cell counts at time points that were not experimentally measured were obtained by regression analysis by fitting a linear model for log-transformed cell counts at day 0 and the end of the treatment (assuming exponential cell growth).

### Example 1

### The In Vitro Effect on Proliferation of Combining the PIK3CA Inhibitor BYL179 and the CDK4/6 Inhibitor LEE011 with the B-Raf Inhibitor Dabrafenib in B-Raf Mutant Colorectal Cancer Cell Lines

To test the effect of the combination of BYL719, LEE011, and dabrafenib on cell proliferation cells were plated in black 384-well microplates with clear bottom (Matrix/Thermo Scientific, Catalog number 4332) in 50 μL media per well at cell densities between 500 and 1250 cells/well (Table 1) and allowed to incubate at 37 degrees, 5% CO₂ for 24 h. After 24 h one 384-well plate per cell line was prepared for cell counting by microscopy (see below) without receiving treatment (=‘baseline’). The other cell plates were treated by transferring 25 nL of the 2000× compound from drug master plates using an ATS acoustic liquid dispenser (ECD Biosystems) and resulting in a final 1× concentration. BYL719 was used over a final concentration range of 13 nM-10 μM, LEE011 was used over a final concentration range of 13 nM-10 μM, and dabrafenib was used over a final concentration range of 1.4 nM-1 μM (7 1:3 dilution steps). In order to assess the effect of the triple combination all individual compounds, all three pair wise combinations (BYL719+LEE011, BYL719+dabrafenib, LEE011+dabrafanic), and the triple combination (BYL719+LEE011+dabrafenib) were tested in the same experiment. Pair wise combinations and the triple combination were tested at a fixed ratio of 1:1 (for drug pairs) and 1:1:1 (for the drug triple) at each dilution resulting in 7 combination conditions per treatment. Additionally, negative controls (DMSO=‘vehicle’) and positive controls (Staurosporine =killing cells, 7-point 1:2 dilution series for a dose range of 16 nM-1 μM) were transferred as treatment controls, and compounds with no efficacy in the cell lines tested were used in combinations with BYL719 and LEE011 as combination controls (combinations that do not exceed the efficacy of the more efficacious single agent=‘non-interacting’ combinations). After compound addition 50 nL of 2 mM CellEvent Caspase-3/7 Green Detection Reagent (ThermoFisher, Catalog number C10423) were added to one of the three replicates using the HP D300 Digital Dispenser (Tecan). Caspase 3/7 induction was measured as a proxy for apoptosis induced by the treatments. Cells were treated for 72 h to 96 h depending on their doubling time (Table 1), and Caspase 3/7 activation was measured every 24 h by microscopy using an InCell Analyzer 2000 (GE Healthcare) equipped with a 4× objective and FITC excitation/emission filters. At the end of the treatment cells were prepared for cell counting by microscopy. Cells were fixed and permeabilised for 45 minutes in 4% PFA (Electron Microscopy Sciences, Catalog number 15714), 0.12% TX-100 (Electron Microscopy Sciences, Catalog number 22140) in PBS (Boston Bioproducts, Catalog number BM-220). After washing cells three times with PBS their DNA was stained for 30 minutes with Hoechst 33342 (ThermoFisher, Catalog number H3570) at a final concentration of 4 μg/mL. Cells were washed three times with PBS and then plates were heat-sealed using a PlateLoc (Agilent Technologies) with aluminum seals (Agilent Technologies, Catalog number 06644-001) and stored at 4° C. until imaging. All cells per well/treatment were captured in a single image by fluorescence microscopy using an InCell Analyzer 2000 (GE Healthcare) equipped with a 4× objective and DAPI excitation/emission filters.

The efficacies of a PIK3CA inhibitor BYL719, a CDK4/6 inhibitor LEE011, and a B-Raf inhibitor dabrafenib were assessed individually and in combination in a total of 6 B-Raf mutant colorectal cancer cell lines, 3 of which were also mutant for PIK3CA (Table 1). BYL719 was effective in the PIK3CA mutant cells with micromolar IC50s, while LEE011 was effective in all but one cell line (ODMS-23) with low micromolar IC50s (FIG. 1 and Table 2). Dabrafenib was effective in all but one cell line (ODMS-23) with nanomolar to low micromolar IC50s (FIG. 1 and Table 2). The triple combination (BYL719+LEE011+dabrafenib) caused synergistic inhibition (according to the HSA model) over the drug pairs in 2/6 cell lines as well as weakly synergistic inhibition in 2/6 cell lines (Table 2). The triple combination does not induce apoptosis (assessed by measuring Caspase 3/7 induction) stronger compared to the pair wise combinations (FIG. 2). Collectively, combined inhibition of PIK3CA, CDK4/6, and B-Raf in B-Raf mutant CRC may provide an effective therapeutic modality capable of improving responses compared to each of the single agents and lead to more durable responses in the clinic.

TABLE 2
Single agent IC50 values for each compound and
synergy z-score measurements for the
combination of LEE011, dabrafenib, and BYL719.

| | IC50 | IC50 | IC50 | Synergy |
|---|---|---|---|---|
| Cell | BYL719 | LEE011 | Dabrafenib | z-score (z_{c}) |
| RKO | 3.9 | 1.5 | 0.24 | 5.8 |
| LS411N | >10 | 2.1 | 0.036 | 3.6 |
| HT-29 | 2.7 | 0.8 | 0.016 | 2.6 |
| LIM2551 | 2.3 | 1.3 | 0.018 | 2.5 |
| COLO-205 | >10 | 1.1 | 0.007 | 1.8 |
| OUMS-23 | >10 | >10 | >1 | −0.2 |

### Example 2

### The In Vitro Effect on Proliferation of Combining the CDK4/6 Inhibitor LEE011 with the B-Raf Inhibitor Dabrafenib in B-Raf Mutant Colorectal Cancer Cell Lines

To test the effect of the combination of LEE011 and dabrafenib on cell proliferation cells were plated in black 384-well microplates with clear bottom (Matrix/Thermo Scientific, Catalog number 4332) in 50 μL media per well at cell densities between 500 and 1250 cells/well (Table 1) and allowed to incubate at 37 degrees, 5% CO₂ for 24 h. After 24 h one 384-well plate per cell line was prepared for cell counting by microscopy (see below) without receiving treatment (=‘baseline’). The other cell plates were treated by transferring 25 nL of the 2000× compound from drug master plates using an ATS acoustic liquid dispenser (ECD Biosystems) and resulting in a final 1× concentration. LEE011 was used over a final concentration range of 13 nM - 10 μM, and dabrafenib was used over a final concentration range of 1.4 nM-1 μM (7 1:3 dilution steps). For the combination of LEE011 with dabrafenib the single agents were combined at a fixed ratio of 1:1 at each dilution resulting in 7 combination treatments. Additionally, negative controls (DMSO=‘vehicle’) and positive controls (Staurosporine=killing cells, 7-point 1:2 dilution series for a dose range of 16 nM-1 μM) were transferred as treatment controls, and compounds with no efficacy in the cell lines tested were used in combinations with LEE011 and dabrafenib as combination controls (combinations that do not exceed the efficacy of the more efficacious single agent=‘non-interacting’ combinations). After compound addition 50 nL of 2 mM CellEvent Caspase-3/7 Green Detection Reagent (ThermoFisher, Catalog number C10423) were added to one of the three replicates using the HP D300 Digital Dispenser (Tecan). Caspase 3/7 induction was measured as a proxy for apoptosis induced by the treatments. Cells were treated for 72 h to 96 h depending on their doubling time (Table 1), and Caspase 3/7 activation was measured every 24 h by microscopy using an InCell Analyzer 2000 (GE Healthcare) equipped with a 4× objective and FITC excitation/emission filters. At the end of the treatment cells were prepared for cell counting by microscopy. Cells were fixed and permeabilised for 45 minutes in 4% PFA (Electron Microscopy Sciences, Catalog number 15714), 0.12% TX-100 (Electron Microscopy Sciences, Catalog number 22140) in PBS (Boston Bioproducts, Catalog number BM-220). After washing cells three times with PBS their DNA was stained for 30 minutes with Hoechst 33342 (ThermoFisher, Catalog number H3570) at a final concentration of 4 μg/mL. Cells were washed three times with PBS and then plates were heat-sealed using a PlateLoc (Agilent Technologies) with aluminum seals (Agilent Technologies, Catalog number 06644-001) and stored at 4° C. until imaging. All cells per well/treatment were captured in a single image by fluorescence microscopy using an InCell Analyzer 2000 (GE Healthcare) equipped with a 4X objective and DAPI excitation/emission filters.

The efficacies of a CDK4/6 inhibitor LEE011 and a B-Raf inhibitor dabrafenib were assessed individually and in combination in a total of 6 B-Raf colorectal cancer cell lines (3 also were mutant for PIK3CA) (Table 1). LEE011 as single agent inhibited the growth of all but one cell line (ODMS-23) with micromolar IC50 values (FIG. 3 and Table 3). Dabrafenib as single agent strongly inhibited the growth of all but one cell line (ODMS-23) with nanomolar to sub-micromolar IC50 values (FIG. 3 and Table 3). The combination treatment caused synergistic inhibition (according to the HSA model) in 5/6 cell lines tested, and with different strengths (Table 3). The combination does not induce apoptosis (assessed by measuring Caspase 3/7 induction) stronger compared to the single agents, which might be a result of the cell-cycle arrest induced after inhibition of CDK4/6 (FIG. 4). Combined inhibition of CDK4/6 and B-Raf in B-Raf mutant colorectal cancer may provide an effective therapeutic modality capable of improving responses compared to each of the single agents and lead to more durable responses in the clinic.

TABLE 3
Single agent IC50 values
for each compound and
synergy z-score measurements for the
combination of LEE011 and dabrafenib.

| | IC50 | IC50 | Synergy |
|---|---|---|---|
| Cell | LEE011 | Dabrafenib | z-score (z_{c}) |
| LS411N | 2.1 | 0.036 | 10.3 |
| HT-29 | 0.8 | 0.016 | 9.6 |
| RKO | 1.5 | 0.24 | 9.5 |
| LIM2551 | 1.3 | 0.018 | 7.5 |
| COLO-205 | 1.1 | 0.007 | 4.4 |
| OUMS-23 | >10 | >1 | −0.1 |

### Example 3

### Synthesis of Methods for Dabrafenib

### Method 1: Dabrafenib (first crystal form)-N-{3-[5-(2-Amino-4-pyrimidinyl)-2-(1,1-dimethylethyl)-1,3 -thiazol-4-yl]-2-fluorophenyl}-2,6-difluorobenzenesulfonamide

A suspension of N-{3-[5-(2-chloro-4-pyrimidinyl)-2-(1,1-dimethylethyl)-1,3-thiazol-4-yl]-2-fluorophenyl}-2,6-difluorobenzenesulfonamide (196 mg, 0.364 mmol) and ammonia in methanol 7M (8 ml, 56.0 mmol) was heated in a sealed tube to 90° C. for 24 h. The reaction was diluted with DCM and added silica gel and concentrated. The crude product was chromatographed on silica gel eluting with 100% DCM to 1:1 [DCM:(9:1 EtOAc:MeOH)]. The clean fractions were concentrated to yield the crude product. The crude product was repurified by reverse phase HPLC (a gradient of acetonitrile:water with 0.1% TFA in both). The combined clean fractions were concentrated then partitioned between DCM and saturated NaHCO₃. The DCM layer was separated and dried over Na₂SO₄. The title compound, N-{3-[5-(2-amino-4-pyrimidinyl)-2-(1,1-dimethylethyl)-1,3-thiazol-4-yl]-2-fluorophenyl}-2,6-difluorobenzenesulfonamide was obtained (94 mg, 47% yield). ¹H NMR (400 MHz, DMSO-d6) δ ppm 10.83 (s, 1 H), 7.93 (d, J=5.2 Hz, 1 H), 7.55-7.70 (m, 1 H), 7.35-7.43 (m, 1 H), 7.31 (t, J=6.3 Hz, 1 H), 7.14-7.27 (m, 3 H), 6.70 (s, 2 H), 5.79 (d, J=5.13 Hz, 1 H), 1.35 (s, 9 H). MS (ESI): 519.9 [M+H]⁺.

### Method 2: Dabrafenib (alternative crystal form)-N-{3-[5-(2-Amino-4-pyrimidinyl)-2-(1,1-dimethylethyl)-1,3-thiazol-4-yl]-2-fluorophenyl}-2,6-difluorobenzenesulfonamide

19.6 mg of N-{3-[5-(2-Amino-4-pyrimidinyl)-2-(1,1-dimethylethyl)-1,3-thiazol-4-yl]-2-fluorophenyl}-2,6-difluorobenzenesulfonamide (may be prepared in accordance with example 58a) was combined with 500 μL of ethyl acetate in a 2-mL vial at room temperature. The slurry was temperature-cycled between 0-40° C. for 48 hrs. The resulting slurry was allowed to cool to room temperature and the solids were collected by vacuum filtration. The solids were analyzed by Raman, PXRD, DSC/TGA analyses, which indicated a crystal form different from the crystal form resulting from Example 58a, above.

### Method 3: Dabrafenib (alternative crystal form, large batch)-N-{3-[5-(2-amino-4-pyrimidinyl)-2-(1,1-dimethylethyl)-1,3-thiazol-4-yl]-2-fluorophenyl}-2,6-d0ifluorobenzenesulfonamide

Step A: methyl 3-{[(2,6-difluorophenyl)sulfonyl]amino}-2-fluorobenzoate

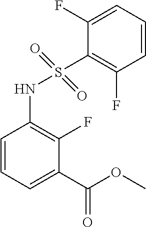 [SMILES: COC(=O)c1cccc(NS(=O)(=O)c2c(F)cccc2F)c1F]

Methyl 3-amino-2-fluorobenzoate (50 g, 1 eq) was charged to reactor followed by dichloromethane (250 mL, 5 vol). The contents were stirred and cooled to ˜15° C. and pyridine (26.2 mL, 1.1 eq) was added. After addition of the pyridine, the reactor contents were adjusted to ˜15° C. and the addition of 2,6-diflurorobenzenesulfonyl chloride (39.7 mL, 1.0 eq) was started via addition funnel. The temperature during addition was kept <25° C. After complete addition, the reactor contents were warmed to 20-25° C. and held overnight. Ethyl acetate (150 mL) was added and dichloromethane was removed by distillation. Once distillation was complete, the reaction mixture was then diluted once more with ethyl acetate (5 vol) and concentrated. The reaction mixture was diluted with ethyl acetate (10 vol) and water (4 vol) and the contents heated to 50-55° C. with stirring until all solids dissolve. The layers were settled and separated. The organic layer was diluted with water (4 vol) and the contents heated to 50-55° C. for 20-30 min. The layers were settled and then separated and the ethyl acetate layer was evaporated under reduced pressure to ˜3 volumes. Ethyl Acetate (5 vol.) was added and again evaporated under reduced pressure to ˜3 volumes. Cyclohexane (9 vol) was then added to the reactor and the contents were heated to reflux for 30 min then cooled to 0° C. The solids were filtered and rinsed with cyclohexane (2×100 mL). The solids were air dried overnight to obtain methyl 3-{[(2,6-difluorophenyl)sulfonyl]amino}-2-fluorobenzoate (94.1 g, 91%).

### Step B: N-{3-[(2-chloro-4-pyrimidinyl)acetyl]-2-fluorophenyl}-2,6-difluorobenzenesulfonamide

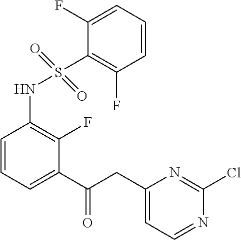 [SMILES: O=C(Cc1ccnc(Cl)n1)c1cccc(NS(=O)(=O)c2c(F)cccc2F)c1F]

Methyl 3-{[(2,6-difluorophenyl)sulfonyl]amino}-2-fluorobenzoate (490 g, 1 equiv.), prepared generally in accordance with Step A, above, was dissolved in THF (2.45 L, 5 vols) and stirred and cooled to 0-3° C. 1M lithium bis(trimethylsilyl)amide in THF (5.25 L, 3.7 equiv.) solution was charged to the reaction mixture followed addition of 2-chloro-4-methylpyrimidine (238 g, 1.3 equiv.) in THF (2.45 L, 5 vols). The reaction was then stirred for 1 hr. The reaction was quenched with 4.5M HCl (3.92 L, 8 vols). The aqueous layer (bootom layer) was removed and discarded. The organic layer was concentrated under reduced pressure to ˜2 L. IPAc (isopropyl acetate) (2.45L) was added to the reaction mixture which was then concentrated to ˜2L. IPAc (0.5L) and MTBE (2.45 L) was added and stirred overnight under N₂. The solids were filtered. The solids and mother filtrate added back together and stirred for several hours. The solids were filtered and washed with MTBE (˜5 vol). The solids were placed in vacuum oven at 50° C. overnight. The solids were dried in vacuum oven at 30° C. over weekend to obtain N-{3-[(2-chloro-4-pyrimidinyl)acetyl]-2-fluorophenyl}-2,6-difluorobenzenesulfonamide (479 g, 72%).

### Step C: N-{3-[5-(2-chloro-4-pyrimidinyl)-2-(1,1-dimethylethyl)-1,3-thiazol-4-yl]-2-fluorophenyl}-2,6-difluorobenzenesulfonamide

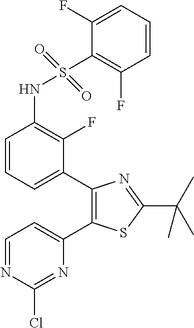 [SMILES: CC(C)(C)c1nc(-c2cccc(NS(=O)(=O)c3c(F)cccc3F)c2F)c(-c2ccnc(Cl)n2)s1]

To a reactor vessel was charged N-{3-[(2-chloro-4-pyrimidinyl)acetyl]-2-fluorophenyl}-2,6-difluorobenzenesulfonamide (30 g, 1 eq) followed by dichloromethane (300 mL). The reaction slurry was cooled to ˜10° C. and N-bromosuccinimide (“NBS”) (12.09 g, 1 eq) was added in 3 approximately equal portions, stirring for 10-15 minutes between each addition. After the final addition of NBS, the reaction mixture was warmed to ˜20° C. and stirred for 45 min .

Water (5 vol) was then added to the reaction vessel and the mixture was stirred and then the layers separated. Water (5 vol) was again added to the dichloromethane layer and the mixture was stirred and the layers separated. The dichloromethane layers were concentrated to ˜120 mL. Ethyl acetate (7 vol) was added to the reaction mixture and concentrated to ˜120 mL. Dimethylacetamide (270 mL) was then added to the reaction mixture and cooled to ˜10° C. 2,2-Dimethylpropanethioamide (1.3 g, 0.5 eq) in 2 equal portions was added to the reactor contents with stirring for ˜5 minutes between additions. The reaction was warmed to 20-25° C. After 45 min, the vessel contents were heated to 75° C. and held for 1.75 hours. The reaction mixture was then cooled to 5° C. and water (270 ml) was slowly charged keeping the temperature below 30° C. Ethyl acetate (4 vol) was then charged and the mixture was stirred and layers separated. Ethyl acetate (7 vol) was again charged to the aqueous layer and the contents were stirred and separated. Ethyl acetate (7 vol) was charged again to the aqueous layer and the contents were stirred and separated. The organic layers were combined and washed with water (4 vol) 4 times and stirred overnight at 20-25° C. The organic layers were then concentrated under heat and vacuum to 120 mL. The vessel contents were then heated to 50° C. and heptanes (120 mL) were added slowly. After addition of heptanes, the vessel contents were heated to reflux then cooled to 0° C. and held for ˜2 hrs. The solids were filtered and rinsed with heptanes (2×2 vol). The solid product was then dried under vacuum at 30° C. to obtain N-{3-[5-(2-chloro-4-pyrimidinyl)-2-(1,1-dimethylethyl)-1,3-thiazol-4-yl]-2-fluorophenyl}-2,6-difluorobenzenesulfonamide (28.8 g, 80%).

### Step D: N-{3-[5-(2-amino-4-pyrimidinyl)-2-(1,1-dimethylethyl)-1,3-thiazol-4-yl]-2-fluorophenyl}-2,6-difluorobenzenesulfonamide

In 1 gal pressure reactor, a mixture of N-{3-[5-(2-chloro-4-pyrimidinyl)-2-(1,1-dimethylethyl)-1,3-thiazol-4-yl]-2-fluorophenyl}-2,6-difluorobenzenesulfonamide (120 g) prepared in accordance with Step C, above, and ammonium hydroxide (28-30%, 2.4 L, 20 vol) was heated in the sealed pressure reactor to 98-103° C. and stirred at this temperature for 2 hours. The reaction was cooled slowly to room temperature (20° C.) and stirred overnight. The solids were filtered and washed with minimum amount of the mother liquor and dried under vacuum. The solids were added to a mixture of EtOAc (15 vol)/water (2 vol) and heated to complete dissolution at 60-70° C. and the aqueous layer was removed and discarded. The EtOAC layer was charged with water (1 vol) and neutralized with aq. HCl to ˜pH 5.4-5.5.and added water (1 vol). The aqueous layer was removed and discarded at 60-70° C. The organic layer was washed with water (1 vol) at 60-70° C. and the aqueous layer was removed and discarded. The organic layer was filtered at 60° C. and concentrated to 3 volumes. EtOAc (6 vol) was charged into the mixture and heated and stirred at 72° C. for 10 min , then cooled to 20° C. and stirred overnight. EtOAc was removed via vacuum distillation to concentrate the reaction mixture to ˜3 volumes. The reaction mixture was maintained at ˜65-70° C. for ˜30mins. Product crystals having the same crystal form as those prepared in Example 58b (and preparable by the procedure of Example 58b), above, in heptanes slurry were charged. Heptane (9 vol) was slowly added at 65-70° C. The slurry was stirred at 65-70° C. for 2-3 hours and then cooled slowly to 0-5° C. The product was filtered, washed with EtOAc/heptane (3/1 v/v, 4 vol) and dried at 45° C. under vacuum to obtain N-{3-[5-(2-amino-4-pyrimidinyl)-2-(1,1-dimethylethyl)-1,3-thiazol-4-yl]-2-fluorophenyl}-2,6-difluorobenzenesulfonamide (102.3 g, 88%).

### Method 4: Dabrafenib (mesylate salt)- N-{3-[5-(2-amino-4-pyrimidinyl)-2-(1,1-dimethylethyl)-1,3-thiazol-4-yl]-2-fluorophenyl}-2,6-difluorobenzenesulfonamide methanesulfonate

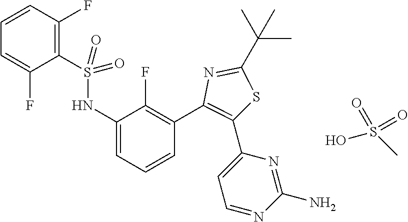

To a solution of N-{3-[5-(2-amino-4-pyrimidinyl)-2-(1,1-dimethylethyl)-1,3-thiazol-4-yl]-2-fluorophenyl}-2,6-difluorobenzenesulfonamide (204 mg, 0.393 mmol) in isopropanol (2 mL), methanesulfonic acid (0.131 mL, 0.393 mmol) was added and the solution was allowed to stir at room temperature for 3 hours. A white precipitate formed and the slurry was filtered and rinsed with diethyl ether to give the title product as a white crystalline solid (210 mg, 83% yield). ¹H NMR (400 MHz, DMSO-d6) δ ppm 10.85 (s, 1 H) 7.92-8.05 (m, 1 H) 7.56-7.72 (m, 1 H) 6.91-7.50 (m, 7 H) 5.83-5.98 (m, 1 H) 2.18-2.32 (m, 3 H) 1.36 (s, 9 H). MS (ESI): 520.0 [M+H]⁺.

### Method 5: Dabrafenib (alternative mesylate salt embodiment)-N-{3-[5-(2-amino-4-pyrimidinyl)-2-(1,1-dimethylethyl)-1,3-thiazol-4-yl]-2-fluorophenyl}-2,6-difluorobenzenesulfonamide methanesulfonate

N-{3-[5-(2-amino-4-pyrimidinyl)-2-(1,1-dimethylethyl)-1,3-thiazol-4-yl]-2-fluorophenyl}-2,6-difluorobenzenesulfonamide (as may be prepared according to example 58a) (2.37 g, 4.56 mmol) was combined with pre-filtered acetonitrile (5.25 vol, 12.4 mL). A pre-filtered solution of mesic acid (1.1 eq., 5.02 mmol, 0.48 g) in H₂O (0.75 eq., 1.78 mL) was added at 20° C. The temperature of the resulting mixture was raised to 50-60° C. while maintaining a low agitation speed. Once the mixture temperature reached to 50-60° C., a seed slurry of N-{3-[5-(2-amino-4-pyrimidinyl)-2-(1,1-dimethylethyl)-1,3-thiazol-4-yl]-2-fluorophenyl}-2,6-difluorobenzenesulfonamide methanesulfonate (1.0%w/w slurried in 0.2 vol of pre-filtered acetonitrile) was added, and the mixture was aged while agitating at a speed fast enough to keep solids from settling at 50-60° C. for 2 hr. The mixture was then cooled to 0-5° C. at 0.25° C./min and held at 0-5° C. for at 6 hr. The mixture was filtered and the wet cake was washed twice with pre-filtered acetonitrile. The first wash consisted of 14.2 ml (6 vol) pre-filtered acetonitrile and the second wash consisted of 9.5 ml (4 vol) pre-filtered acetonitrile. The wet solid was dried at 50° C. under vacuum, yielding 2.39 g (85.1% yield) of product.

1. A pharmaceutical combination comprising:
(a) a first compound having the structure of Formula (I): 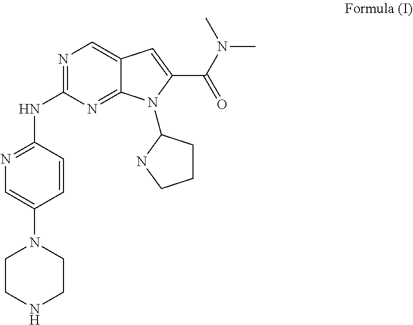 [SMILES: CN(C)C(=O)c1cc2cnc(Nc3ccc(N4CCNCC4)cn3)nc2n1C1CCCN1]
or a pharmaceutically acceptable salt or solvate thereof, and
(b) a second compound having the structure of Formula (II): 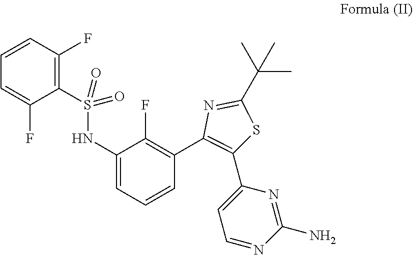 [SMILES: CC(C)(C)c1nc(-c2cccc(NS(=O)(=O)c3c(F)cccc3F)c2F)c(-c2ccnc(N)n2)s1]
or a pharmaceutically acceptable salt or solvate thereof.

2. The pharmaceutical combination of claim 1, wherein the compound having the structure of Formula (I), or a pharmaceutically acceptable salt or solvate thereof, and the compound having the structure of Formula (II), or a pharmaceutically acceptable salt or solvate thereof, are in the same formulation.

3. The pharmaceutical combination of claim 1, wherein the compound having the structure of Formula (I), or a pharmaceutically acceptable salt or solvate thereof, and the compound having the structure of Formula (II), or a pharmaceutically acceptable salt or solvate thereof, are in separate formulations.

4. The pharmaceutical combination of claim 1, wherein the combination is for simultaneous or sequential administration.

5. The pharmaceutical combination of claim 1, further comprising a third compound having the structure of Formula (III): 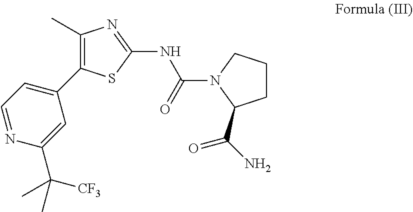 [SMILES: Cc1nc(NC(=O)N2CCC[C@H]2C(N)=O)sc1-c1ccnc(C(C)(C)C(F)(F)F)c1]
or a pharmaceutically acceptable salt or solvate thereof.

6. The pharmaceutical combination of claim 5, wherein the compound having the structure of Formula (I), or a pharmaceutically acceptable salt or solvate thereof, the compound having the structure of Formula (II), or a pharmaceutically acceptable salt or solvate thereof, and the compound having the structure of Formula (III), or a pharmaceutically acceptable salt or solvate thereof, are in the same formulation.

7. The pharmaceutical combination of claim 5, wherein the compound having the structure of Formula (I), or a pharmaceutically acceptable salt or solvate thereof, the compound having the structure of Formula (II), or a pharmaceutically acceptable salt or solvate thereof, and the compound having the structure of Formula (III), or a pharmaceutically acceptable salt or solvate thereof, are in 2 or 3 separate formulations.

8. The pharmaceutical combination of claim 5, wherein the combination is for simultaneous or sequential administration.

9. The pharmaceutical combination of claim 1, wherein the first compound is the succinate salt of the compound having the structure of Formula (I).

10. A method for the treatment or prevention of cancer in a subject in need thereof, comprising administering to the subject a therapeutically effective amount of a pharmaceutical combination of claim 1.

11. The method of claim 10, wherein the cancer is selected from the group consisting of melanoma, lung cancer (including non-small-cell lung cancer (NSCLC)), colorectal cancer (CRC), breast cancer, kidney cancer, renal cell carcinoma (RCC), liver cancer, acute myelogenous leukemia (AML), myelodysplastic syndromes (MDS), thyroid cancer, pancreatic cancer, neurofibromatosis and hepatocellular carcinoma.

12. The method of claim 10, wherein the cancer is colorectal cancer.

13. The method of claim 10, wherein the cancer is characterized by one or more of a B-Raf mutation, B-Raf V600E mutation, PIK3CA mutation and PIK3CA overexpression.

14. The pharmaceutical combination of claim 1, for use in the treatment or prevention of cancer.

15. The pharmaceutical combination of claim 1, for use in the manufacture of a medicament for the treatment or prevention of cancer.

16. The pharmaceutical combination of claim 14 claim 14, wherein the cancer is selected from the group consisting of melanoma, lung cancer (including non-small-cell lung cancer (NSCLC)), colorectal cancer (CRC), breast cancer, kidney cancer, renal cell carcinoma (RCC), liver cancer, acute myelogenous leukemia (AML), myelodysplastic syndromes (MDS), thyroid cancer, pancreatic cancer, neurofibromatosis and hepatocellular carcinoma.

17. The pharmaceutical combination of claim 16, wherein the cancer is colorectal cancer.

18. The pharmaceutical combination of claim 14, wherein the cancer is characterized by one or more of a B-Raf mutation, B-Raf V600E mutation, PIK3CA mutation and PIK3CA overexpression.

19. Use of a pharmaceutical combination of claim 1 for the manufacture of a medicament for the treatment or prevention of cancer.

20. Use of a pharmaceutical combination of claim 1 for the treatment or prevention of cancer.

21. The use of claim 19, wherein the cancer is selected from the group consisting of melanoma, lung cancer (including non-small-cell lung cancer (NSCLC)), colorectal cancer (CRC), breast cancer, kidney cancer, renal cell carcinoma (RCC), liver cancer, acute myelogenous leukemia (AML), myelodysplastic syndromes (MDS), thyroid cancer, pancreatic cancer, neurofibromatosis and hepatocellular carcinoma.

22. The use of claim 21, wherein the cancer is colorectal cancer.

23. The use of claim 19, wherein the cancer is characterized by one or more of a B-Raf mutation, B-Raf V600E mutation, PIK3CA mutation and PIK3CA overexpression.

24. A pharmaceutical composition comprising:
(a) a first compound having the structure of Formula (I): 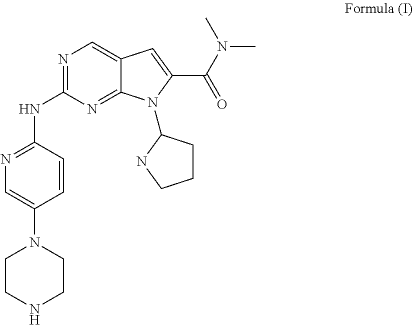 [SMILES: CN(C)C(=O)c1cc2cnc(Nc3ccc(N4CCNCC4)cn3)nc2n1C1CCCN1]
or a pharmaceutically acceptable salt thereof, and
(b) a second compound having the structure of Formula (II): 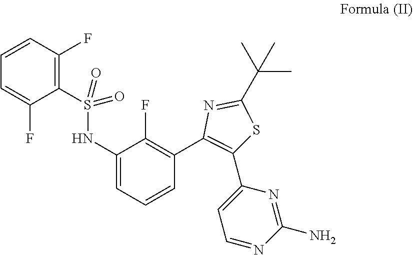 [SMILES: CC(C)(C)c1nc(-c2cccc(NS(=O)(=O)c3c(F)cccc3F)c2F)c(-c2ccnc(N)n2)s1]
or a pharmaceutically acceptable salt thereof.

25. The pharmaceutical composition of claim 24, further comprising a third compound having the structure of Formula (III): 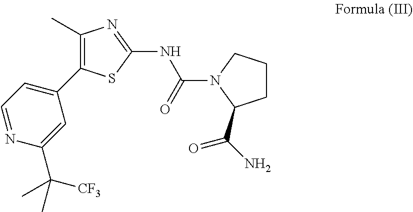 [SMILES: Cc1nc(NC(=O)N2CCC[C@H]2C(N)=O)sc1-c1ccnc(C(C)(C)C(F)(F)F)c1]
or a pharmaceutically acceptable salt thereof.

26. The pharmaceutical composition of claim 24, further comprising one or more excipients.
